(19) United States
(12) Patent Application Publication
(10) Pub. No.: US 2007/0270662 A1
(43) Pub. Date: Nov. 22, 2007

(54) APPARATUS AND METHOD FOR MONITORING END USER'S HEALTH STATUS VIA A NETWORK

(76) Inventor: Yung-Lung Chen, Hsinchuang (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 11/419,289

(22) Filed: May 19, 2006

(51) Int. Cl. A61B 5/00

(52) U.S. Cl. 600/300

(57) ABSTRACT

An apparatus for monitoring end user's health status via a network comprises at least one system server or a personal server connected to a plurality of monitoring devices and a plurality of medical organization servers. The monitoring devices, the system server or the personal sever, and the medical organization servers are connected by wireless transmission and network. When an alarm message is sent out from the end user, the personal server will send this information to predetermined telephone or to the medical organization server automatically, the telephone can be family or relative's mobile phone, or the telephone of a medical or rescue organization. Therefore, the end user can get an immediate treatment.

### BACKGROUND OF THE INVENTION

1. Field of the Invention

The present invention relates to an apparatus and a method for monitoring end user's health status, and more particularly to an apparatus and a method for monitoring end user's health status via a network.

2. Description of the Prior Art

Computer network has come into existence in many respects, however, it has not become popular in medical and rescue application. For example, when a patient suffers from a sudden disease, and the doctors and nurses don't have enough time to take care of him, a high-income family might hire a person to take care of the patient, and time, money and labor will be wasted. Some diseases cannot be diagnosed by manual examination but must be examined by medical instrument. Besides, a low-income family is usually not able to afford the cost of hiring a person to effectively take care of the patient, and if the patient loses consciousness due to a sudden disease, such as heart attack or hypertension, he/she cannot receive a treatment in time, and this will endanger the patient's life.

The present invention has arisen to mitigate and/or obviate the afore-described disadvantages.

### SUMMARY OF THE INVENTION

The primary objective of the present invention is to provide an apparatus and a method for monitoring end user's health status via a network, when an alarm message is sent out from the end user, the system server will send this information to the medical organization server immediately, and send out rescue immediately.

The secondary objective of the present invention is to provide an apparatus and a method for monitoring end user's health status via a network, when an alarm message is sent out from the end user, the personal server will send this information to the predetermined telephone automatically, the telephone can be family or relative's mobile phone, or the telephone of a medical or rescue organization, and send out rescue immediately.

Another objective of the present invention is to reduce the number of nurses taking care of the patient and prevent the manmade mistakes, and the accident caused by the manmade mistakes.

Yet another objective of the present invention is to provide a self-rescue function to the end user. When there is no nurse around, the monitoring device worn on the user will monitor the health status of the end user and the information of the health status can be sent out in time.

The present invention is characterized in that the monitoring devices, the system server, and the medical organization servers are connected by wireless transmission and network. By such arrangements, the function of monitoring the end user's health status, location, the information sending can be obtained, and thus the end user can receive the treatment in time.

The present invention is also characterized in that the personal sever, the monitoring devices, and the relative telephones are connected via wireless transmission, network, or Internet. By such arrangements, the function of monitoring the end user's health status, location, the information sending can be obtained, and thus the end user can receive the treatment in time.

### BRIEF DESCRIPTION OF THE DRAWINGS

FIG. 1 is a block diagram of showing an apparatus for monitoring end user's health status via a network in accordance with the present invention;

FIG. 2 is a framework view of showing the apparatus for monitoring end user's health status via a network in accordance with the present invention;

FIG. 3 is a flow chart view of showing the method for monitoring end user's health status via a network in accordance with the present invention;

FIG. 4 is an illustrative view of showing the method for monitoring end user's health status via a network in accordance with the present invention; and

FIG. 5 is another illustrative view of showing the method for monitoring end user's health status via a network in accordance with the present invention.

### DETAILED DESCRIPTION OF THE PREFERRED EMBODIMENTS

The present invention will be more clear from the following description when viewed together with the accompanying drawings, which show, for purpose of illustrations only, the preferred embodiment in accordance with the present invention.

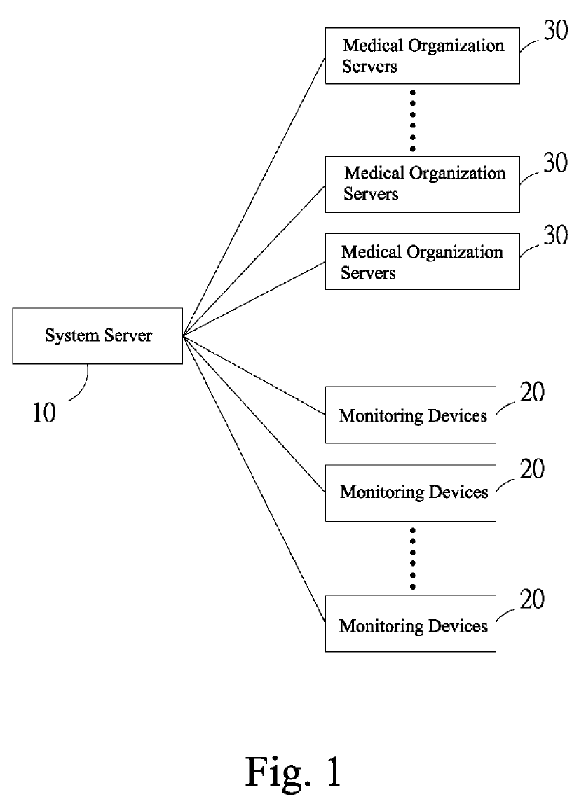

Referring to FIG. 1, an apparatus for monitoring end user's health status via a network in accordance with the present invention comprises a system server 10 connected to a plurality of monitoring devices 20 and a plurality of medical organization servers 30.

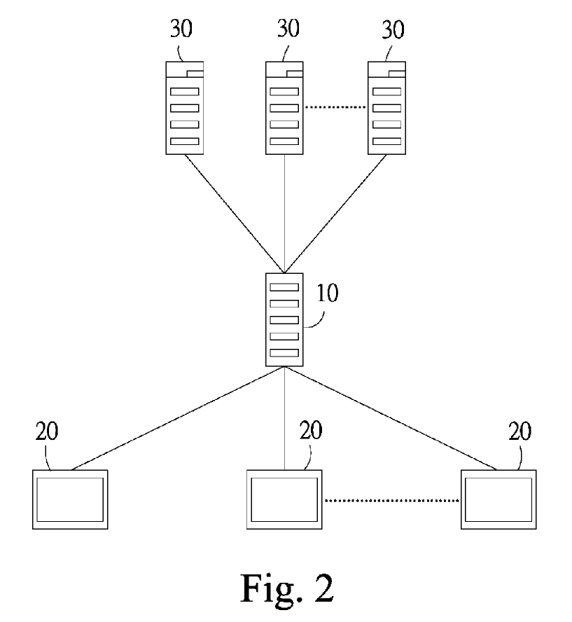

As shown in FIGS. 1 and 2, each end user is equipped with a monitoring device 20, and the monitoring devices 20 are wirelessly connected to the system server 10 (via radio wave, infrared, microwave, or satellite signal). And the medical organization serves 30 are connected to the system server 10 to form a network.

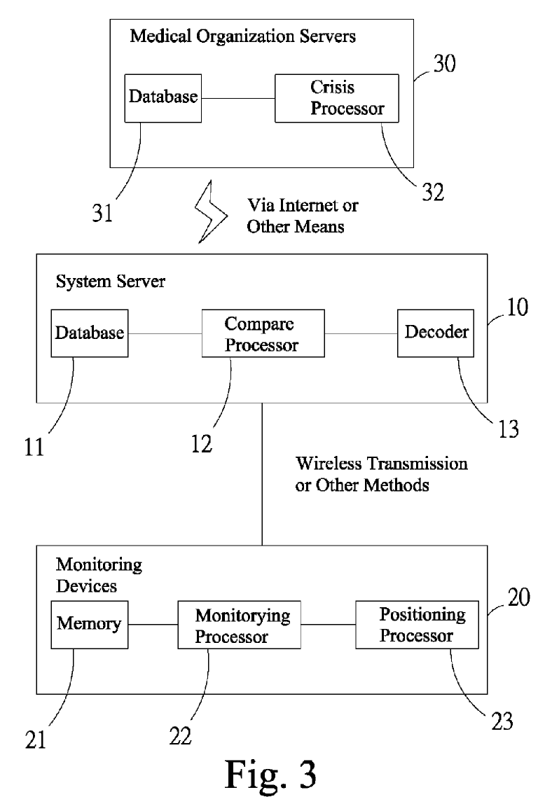

As shown in FIGS. 1 and 3, the system server 10 includes at least one database 11, one decoder 13, and one compare processor 12. The basic data of each end user is saved in the database 11, including at least a personal data, such as name, date of birth, ID number, registered hospital, address and phone number, and a health status information, such as a sample of normal value of blood pressure, pulse, and heartbeat. The decoder 13 serves to receive and decode the identification code from the monitoring device 20, and then provide to the compare processor 12. The compare processor 12 serves to receive information data from the end user and to determine whether the user is health or not by comparing the information data with the corresponding basic data stored in the database 11. The monitoring device 20 includes at least one inner memory 21, one monitoring processor 22 and a positioning processor 23. The basic data of a specific end user is saved in the inner memory 21, including personal data, such as name, date of birth, ID number, registered hospital, address and phone number, a health status information, and a health status information, such as a sample of normal value of blood pressure, pulse, and heartbeat. In the inner memory 21 can also be saved a group of identification code corresponding to the system server 10. The monitoring processor 22 serves to monitor the blood pressure, pulse and heartbeat of the end user. The positioning processor 23 determines the position of the end user by coordination location and then converts it into a readable location name. And the information is transmitted wirelessly and intermittently via satellite within an appropriate time period. The medical organization server 30 includes at least one database 31 and a crisis processor 32. The basic data of all the end users is saved in the database 31, including at least a personal data, such as name, date of birth, ID number, registered hospital, address and phone number, a health status information, a health status information, such as a sample of normal value of blood pressure, pulse, and heartbeat, and medical record data. The crisis processor 32, via network, receives the information sent to the system server 10 from the monitoring device 20. The medical organization server 30 can also be connected to other medical organization servers 30 or rescue facilities (such as Fire Company) via the internet.

The abovementioned monitoring device 20 can be worn on the wrist, and the information is preferably intermittently transmitted every 3 seconds.

As shown in FIG. 3, when the monitoring device 20 worn by the end user is activated, the monitoring processor 22 starts to monitor the blood pressure, pulse and heartbeat of the end user, and the positioning processor 23 determines the position of the end user by satellite positioning method, and then the position information is saved in the inner memory 21. The identification code and the basic data are permanently saved in the inner memory 21, and other information is temporarily saved. After the blood pressure, pulse and heartbeat of the end user monitored by the monitoring processor 22, and the location information detected by the positioning processor 23 are transmitted to the system server 10, the previous temporarily saved data in the system server 10 will be covered, namely, the temporarily saved data in the system server 10 is updated every three seconds. When the system server 10 receives the data (including the basic data, the identification code, the blood pressure, the pulse and the heartbeat) from the monitoring device 20, the data will be by the decoder 13. The compare processor 12 then starts to compare the decoded data with the basic information saved in the database 11 and determine whether the health value of the end user is within the normal range, and if the detected health value is not within the normal range, the system server 10 will send the basic information obtained by the monitoring device 20 via internet to the registered hospital previously saved in the database 11. The crisis processor 32 of the medical organization server 30 receives this information and compares it with the corresponding data selected from the database 31, so that the registered hospital can rescue the patient (the end user) within the shortens time, besides, the medical organization server 30 can also ask for aid through the internet, asking the hospital, medical facilities, fire department nearest the patient to arrange a rescue operation as soon as possible, and thus the patient can get help within the shortest time.

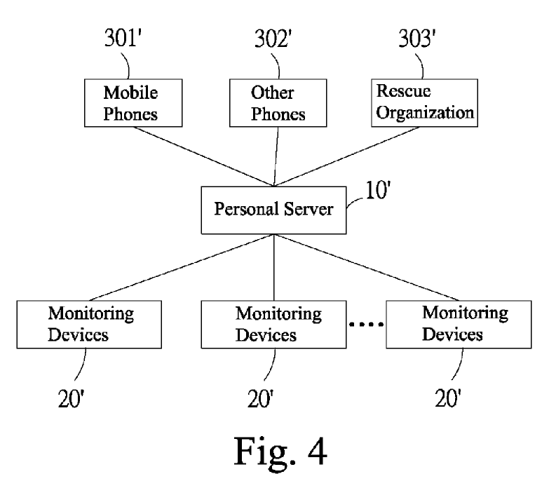

As shown in FIG. 4, a personal server 10′ is connected to a plurality of monitoring devices 20′, a plurality of mobile phones 301′, other phones 302′ or phones 303′ of rescue facilities.

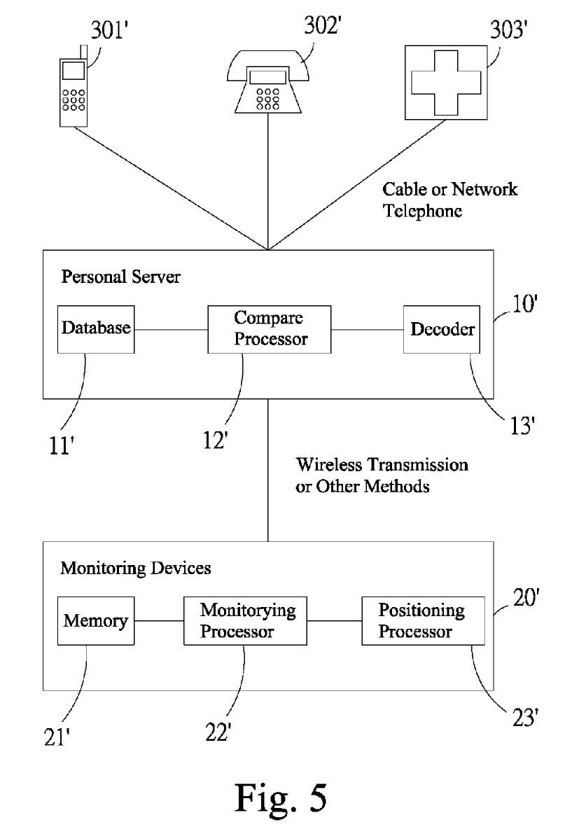

As shown in FIGS. 4 and 5, each end user wears a monitoring device 20′, and the monitoring devices 20′ are wirelessly connected to the personal server 10′ (via radio wave, infrared, microwave, or satellite signal). And the personal server 10′, the monitoring devices 20′, the mobile phones 301′, the phones 302′ or phones 303′ of the rescue facilities are connected together to form a network.

For prevention of transmission interruption, the data is preferably transmitted from the monitoring devices 20 and 20′ intermittently every 3 seconds. Even if the information transmission is interrupted 3 seconds ago, the system server 10 or the personal server 10′ can be programmed in a such manner that the compare processors 12, 12′ will send the detected information prior to the interruption to the medical organization server 30 or the mobile phone 301′, the phones 302′ or phones 303′ of the rescue facilities, under condition that they don't receive continuous signals from the monitoring devices 20, 20′ within a certain period of time. This method can search the position of the end user by reducing the scope, and the position can be controlled within the smallest range.

While we have shown and described various embodiments in accordance with the present invention, it is clear to those skilled in the art that further embodiments may be made without departing from the scope of the present invention.

What is claimed is:

1. An apparatus for monitoring end user's health status via a network comprises at least one system server connected to a plurality of monitoring devices and a plurality of medical organization servers; wherein
the system server includes at least one database, one decoder, and one compare processor, basic data of a plurality of end users is saved in the database, the decoder serves to receive and decode identification code from the monitoring devices, and then provide to the compare processor, the compare processor serves to receive information data from the monitoring devices and to determine whether the end user is health or not by comparing the information data with the corresponding basic data stored in the database;
the monitoring devices each includes at least one inner memory, one monitoring processor and one positioning processor, a basic data of a specific end user is saved in the inner memory, a group of identification code corresponding to the system server is saved in the inner memory, the monitoring processor serves to monitor blood pressure, pulse and heartbeat of the end user, the positioning processor determines the position of the end user by coordination location and then converts it into a readable location name, and these information is wirelessly and intermittently transmitted to the system server via satellite positioning within an appropriate time period;
the medical organization servers each includes at least one database and a crisis processor, the basic data of the plurality of end users is saved in the database of the medical organization servers, the crisis processor, via network, receives information sent to the system server from the monitoring devices.

2. The apparatus for monitoring end user's health status via a network as claimed in claim 1, wherein the basic data of the plurality of end users saved in the database includes at least a personal data, such as name, date of birth, ID number, registered hospital, address and phone number, and a health status information, such as a sample of normal value of blood pressure, pulse, and heartbeat.

3. The apparatus for monitoring end user's health status via a network as claimed in claim 1, wherein the basic data of a specific end user saved in the inner memory includes at least a personal data, such as name, date of birth, ID number, registered hospital, address and phone number, and a health status information, such as a sample of normal value of blood pressure, pulse, and heartbeat.

4. The apparatus for monitoring end user's health status via a network as claimed in claim 1, wherein the basic data of the plurality of end users saved in the database of the medical organization servers includes at least a personal data, such as name, date of birth, ID number, registered hospital, address and phone number, and a health status information, such as a sample of normal value of blood pressure, pulse, and heartbeat, and a case record.

5. The apparatus for monitoring end user's health status via a network as claimed in claim 1, wherein the medical organization servers are also allowed to be connected to other medical organization servers or rescue facilities through internet.

6. The apparatus for monitoring end user's health status via a network as claimed in claim 1, wherein the monitoring devices are wirelessly connected to the system server via radio wave, infrared, microwave, or satellite signal, and the medical organization serves are connected to the system server to form a network.

7. The apparatus for monitoring end user's health status via a network as claimed in claim 1, wherein the monitoring devices are worn on the wrist.

8. The apparatus for monitoring end user's health status via a network as claimed in claim 1, wherein the information is intermittently transmitted from the monitoring devices to the system server every three seconds.

9. The apparatus for monitoring end user's health status via a network as claimed in claim 1, wherein the identification code and the basic data in the inner memory are permanently saved, and other information is temporarily saved, and after the blood pressure, pulse and heartbeat of the end user monitored by the monitoring processor, and the location information detected by the positioning processor are intermittently transmitted to the system server every three seconds, the previous temporarily saved data in the system server will be covered, namely, the temporarily saved data in the system server is updated every three seconds.

10. An apparatus for monitoring end user's health status via a network comprises at least one personal server connected to a plurality of monitoring devices, a plurality of mobile phones, other phones or phones of rescue facilities;
the personal server includes at least one database, one decoder, and one compare processor, basic data of a plurality of end users is saved in the database, the decoder serves to receive and decode identification code from the monitoring devices, and then provide to the compare processor, the compare processor serves to receive information data from the monitoring devices and to determine whether the end user is health or not by comparing the information data with the corresponding basic data stored in the database;
the monitoring devices each includes at least one inner memory, one monitoring processor and one positioning processor, a basic data of a specific end user is saved in the inner memory, a group of identification code corresponding to the personal server is saved in the inner memory, the monitoring processor serves to monitor blood pressure, pulse and heartbeat of the end user, the positioning processor determines the position of the end user by coordination location and then converts it into a readable location name, and these information is wirelessly and intermittently transmitted to the personal server via satellite positioning within an appropriate time period;
if the health status of the end user obtained by the personal server is not within normal range, the personal server will contact the mobile phones, the other phones or the phones of rescue facilities via network or network telephone, so as to rescue the end user as soon as possible.
